(19) United States
(12) Patent Application Publication
(10) Pub. No.: US 2019/0142829 A1
(43) Pub. Date: May 16, 2019

(54) AMELIORATION OF ANGELMAN'S SYNDROME DEFICIENCIES

(71) Applicant: Neuralstem, Inc., Germantown, MD (US)

(72) Inventor: Karl K. JOHE, Hallandale Beach, FL (US)

(73) Assignee: Neuralstem, Inc., Germantown, MD (US)

(21) Appl. No.: 15/961,419

(22) Filed: Apr. 24, 2018

Related U.S. Application Data
(63) Continuation-in-part of application No. PCT/US2017/061187, filed on Nov. 10, 2017.

(51) Int. Cl. A61K 31/506; A61P 25/28; A61P 25/00; A61B 5/00; A61B 5/16

(52) U.S. Cl. CPC A61K 31/506; A61P 25/28; A61P 25/00; A61B 5/168; A61B 5/4088; A61B 5/4082; A61B 5/4848

(57) ABSTRACT

Treatment with 2-amino substituted nicotinamides or their salts ameliorates the cognitive and motor dysfunction associated with Angelman Syndrome (AS).

### CROSS-REFERENCE TO RELATED APPLICATION

This application is a continuation-in-part of PCT/US2017/061187, filed 10 Nov. 2017, the contents of which are incorporated herein by reference.

### TECHNICAL FIELD

The invention relates to treatment of subjects who have Angelman Syndrome (AS) with compounds that ameliorate the cognitive deficits including memory associated with this condition. These compounds also ameliorate motor deficits in these subjects. More particularly, it concerns the use of 2-amino substituted nicotinamides for this purpose.

### BACKGROUND ART

Angelman syndrome (AS) is a maternally inherited disease in which a proteosomal processing enzyme, Ube3A, that is important for synaptic plasticity during learning and memory in development and in adults, is deleted or mutated, resulting in developmental arrest of CNS functions. The clinical manifestations include severe mental retardation, periods of epileptic seizures, ataxia, hyperactivity, and disordered sleep, which last for life-time. Currently there is no effective treatment to treat the underlying synaptic dysfunction.

A family of U.S. granted patents, represented by, for example, U.S. Pat. No. 8,362,262, discloses low molecular weight compounds that are capable of stimulating neuronal growth, and are able to permeate the blood-brain barrier (BBB). Subsequently, it was found that certain 2-amino-substituted nicotinamides were useful in treating depression, in particular, major depressive disorder in humans as described in PCT publication WO2015/195567, and corresponding U.S. Pat. No. 9,572,807. Oral administration of these compounds in such treatments is disclosed. However, nothing in these documents suggests that deficits in cognitive function or in motor function associated with AS can be ameliorated by these compounds despite their known ability to stimulate neuronal growth, and their ability to improve self-perceived cognition in depressed patients.

In work by applicant, hippocampal slices from normal, healthy, young adult mice were treated with various concentrations of NSI-189 (100 nM to 10 μM) for various times up to 3.5 h, before applying theta burst stimulation (TBS) to elicit long-term potentiation (LTP) in field CA1. These treatments resulted in statistically significant enhancement of the normal LTP level. This effect could be inhibited by inhibitors of protein-synthesis or transcription, demonstrating that NSI-189 can directly act on hippocampal excitatory circuitry to enhance its synaptic response to stimuli (Liu et al, Society for Neuroscience Abstract 2016).

Further, in a mouse model of Angelman syndrome, carrying the same maternal Ube3A deletion affected in human AS patients, the normal LTP is absent. Surprisingly, a transient treatment of the hippocampal brain slices prepared from young adult Angelman mice with NSI-189 for 3.5 hours could restore the LTP activity to normal level (Liu et el, Society for Neuroscience Abstract 2016).

### DISCLOSURE OF THE INVENTION

It has now been found that certain 2-amino-substituted nicotinamides are especially useful in prevention and/or repair of various aspects of cognitive and motor skill impairments that are associated with AS.

Accordingly, in one aspect, the invention is directed to a method to ameliorate the cognitive deficiencies and/or motor skill deficits associated with AS by administering to a subject in need of such amelioration, a pharmaceutical composition wherein the active ingredient is a 2-amino-substituted nicotinamide or a pharmaceutically acceptable salt thereof in some embodiments the phosphate salt. In particular, the 2-amino-substituted nicotinamide is of the formula:

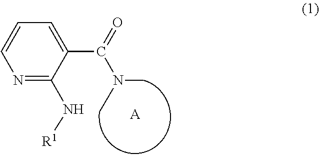

wherein R¹ is an alkyl of 3-8C and ring A is a 5- or 6-membered saturated ring optionally including an additional nitrogen which is unsubstituted or substituted with an additional nitrogen-containing substituent or a ring-opened form thereof.

Particular exemplified compounds include those of formula (2)

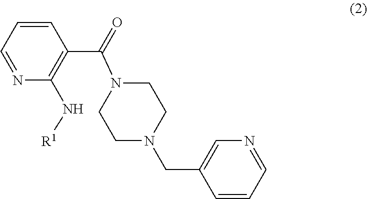

or formula (3)

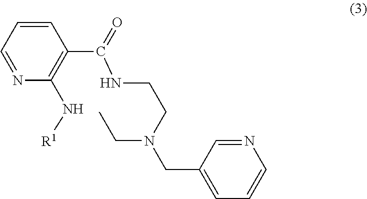

wherein R¹ is a branched alkyl group of 3-5C or of formula (4)

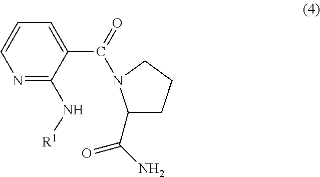

wherein R¹ is an alkyl group comprising a 5- or 6-membered ring.

In one embodiment, the compound of formula (2) is employed as a pharmaceutically acceptable salt, in particular the phosphate salt, and also, in particular, wherein R¹ is isoamyl.

### BRIEF DESCRIPTION OF DRAWINGS

FIG. 1 shows the results of administering NSI-189 to AS and wild type mice on performance in a fear-conditioning test.

FIG. 2 shows the results of administering NSI-189 to AS and wild type mice on long-term potentiation (LTP) assay on hippocampal slices post mortem.

FIG. 3 shows the results of administering NSI-189 to AS and wild type mice on motor skills in a Hanging Wire test.

### MODES OF CARRYING OUT THE INVENTION

The methods of the invention are directed to ameliorating the cognitive deficiencies and/or motor skill deficiencies associated with AS.

The active agents useful in the method of the invention have the general formula (1) noted above wherein R¹ is an alkyl of 3-8C and ring A is a 5 or 6 membered saturated ring optionally including an additional nitrogen or a ring-opened form thereof. Thus, R¹ may be, in formula (1), a straight or branched chain alkyl group of at least 3C, such as isopropyl, secondary butyl, n butyl, isoamyl, sec-amyl, hexyl, isohexyl and the like or comprise a saturated ring. Preferably in formula (2) or (3), R¹ is a branched alkyl of 3-5C and, in formula (4), R¹ comprises a 5- or 6-membered saturated ring. Preferred embodiments of ring A are a piperidine or piperazine ring or ring opened forms thereof or a pyrrolidine ring.

Ring A may also be substituted with at least an additional nitrogen-containing substituent, including a substituent that comprises an additional pyridine ring such as pyridyl methyl, or pyridyl ethyl or is a simpler substituent such as a carboxamide. Preferred forms of ring A are shown in formulas (2), (3) and (4) above along with appropriate substituents.

The compounds of the invention are typically, but not always, administered in the form of their pharmaceutically acceptable salts such as halides, maleates, succinates, nitrates and the like. Particularly favored are phosphate salts.

The compounds of the invention are formulated in standard pharmaceutical formulations such as those found in Remington's Pharmaceutical Sciences, latest edition, Mack Publishing Co., Easton, Pa. and include formulations for oral administration and parenteral administration. The compounds may be administered orally in the form of tablets, capsules or in formulations that are administered as syrups or any other standard formulation. The compounds may also be administered parenterally, including intraperitoneal (i.p.) and intravenously (IV). In some instances, the formulations may be designed for delayed release or may be designed for more instantaneous delivery. A variety of formulations that would be suitable for the compounds of the invention is known in the art and is subject to the decision of the practitioner with regard to route of administration.

Dosage levels also depend on the judgment of the practitioner, but are generally in the range of 0.01 mg/kg to 1 2 g/kg.

In general, the subjects of the treatment will be humans, although it is useful to employ laboratory animals as well in order to assess appropriate dosages, routes of administration and formulations. Thus, the subjects of the invention include not only humans, but laboratory research animals such as rabbits, rats, mice and the like. In some instances, other mammalian subjects may be appropriate such as in veterinary contexts where the subject may be ovine, bovine or equine or the subject may be a companion animal such as dog or cat.

In some embodiments, the method of treatment further includes a concomitant diagnostic procedure whereby the effect of the treatment is evaluated at various timepoints during administration and/or after administration of the compositions of the invention. These evaluations include evaluation of, for example, novel place recognition, novel object recognition, object and place recognition and recognition of temporal order. The analyses may also include fear conditioning. Many tests are known in the art both for cognitive skills and motor skills, some appropriate for humans and others for laboratory models. Any appropriate such test may be used.

A particularly useful diagnostic for cognition in humans is measurement by CogScreen, a computer-administered cognitive test battery required by the U.S. Federal Aviation Administration (FAA) for evaluation of the neurocognitive functioning of pilots and which has also played a key role in the FDA drug approval and labeling process (CogScreen LLC, St Petersburg, FL). This includes analysis of Shifting Attention Test-Arrow Color Accuracy a measure of executive functioning; Shifting Attention Test-Arrow Direction Reaction Time Correct, a measure of attention; Symbol Digit Coding-Delayed Recall Accuracy, a measure of memory and Shifting Attention Test-Instruction Number Incorrect, which is a measure of working memory. One or a combination of these aspects or a subset thereof may be employed.

Other tests include the fear-conditioning test and other tests appropriate for laboratory models are illustrated below.

As shown in the Examples below, the effect of NSI-189 was tested on hippocampal synaptic plasticity. LTP is a widely accepted model of synaptic plasticity. This also serves a biomarker of memory in animal studies intended to model memory formation/processing in human brain. The LTP assay per se does not infallibly represent cognitive functioning of intact brain in a whole live animal, whether normal or diseased. In the Angelman mouse model, for example, genetic replacement of Ube3A protein in the adult brain could restore LTP but not the neurobehavioral deficits. However, while LTP cannot be used alone to predict potential therapeutic efficacy of a drug on cognitive impairment specifically or brain function generally, it shows a probability of this result. Thus, the results in the Example below relating to fear-conditioning are significant.

Motor skills in laboratory models may be tested by the hanging wire test and/or by the elevated body swing test (EBST) and/or by the Bederson test illustrated below. Motor skills in humans may be tested by the Bruininks Motor Ability Test (BMAT) or a battery of motor skills tests found on the web at ukk instituutti or any of a number of recognized tests. Any suitable test may be used.

The frequency of administration and dosage schedules is also dependent on the practitioner and the dose may be chronic and on a daily basis, weekly basis or more frequent, or a single dosage may suffice. In typical protocols, the compositions of the invention are administered orally daily for a predetermined time period which may be weeks or months or on a chronic basis. Dosage may be altered or stopped depending on evaluation of the subject as described above. The compounds of the invention may also be administered in combination with other active agents either in the same composition or sequentially.

The following examples illustrate, but do not limit the invention,

### EXAMPLE 1

### Mouse Model of Angelman's Syndrome

Four groups (n=4/5 per group) of young adult mice (age ˜3 months) were administered 30mg/kg of the phosphate salt of NSI-189 i.p. daily for 13-15 days for behavior assays and for 18 days prior to termination followed by in vitro LTP assays or administered only vehicle. The groups included
(1) mice with Angelman syndrome (AS) treated with NSI-189,
(2) mice with Angelman syndrome (AS) treated with vehicle,
(3) wild-type littermates treated with NSI-189, and
(4) wild-type littermates treated with vehicle.

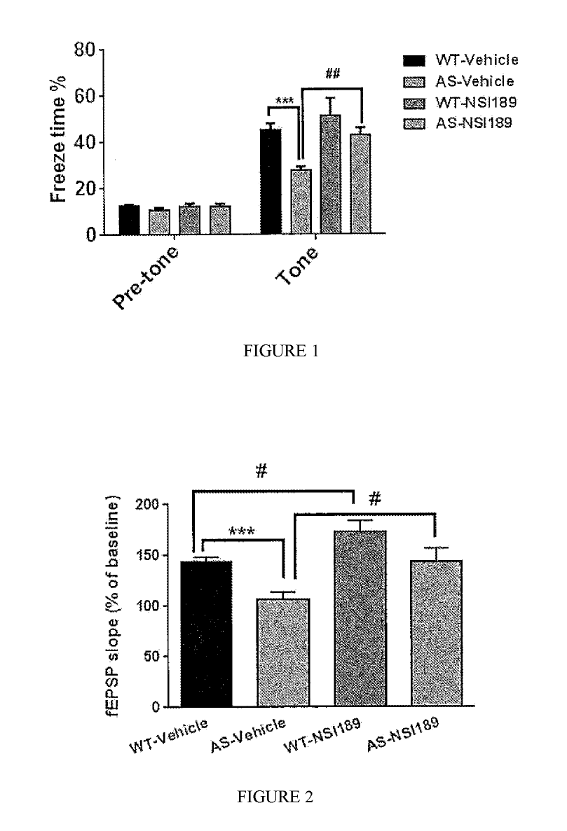

The behavior assay conducted was fear-conditioning test as diagnostic of hippocampal memory function. As shown in FIG. 1, AS mice treated only with vehicle exhibited significantly shortened freezing time following ring-tone, a measure of memory deficit, compared to wild-type mice treated with vehicle. After 2 weeks of treatment with NSI-189 this memory deficit was diminished (p<0.01).

Subsequent analysis of post-mortem hippocampal slices showed restoration of LTP in AS mice treated with NSI-189 vs. vehicle (p<0.05) (FIG. 2). LTP was also improved by administration of NSI-189 in wild type mice.

The wire hang test evaluates muscle strength as well as motor coordination. The set up includes a wire (0.2 cm in diameter×54.5 cm in length) stretched horizontally between two poles and elevated from a padded surface. The test starts when the mouse hangs onto the wire from its two forelimbs and lasts for 120 sec maximum. The time before falling, or the maximum score of 120 sec if no fall occurred, is recorded for each animal.

Four groups 5 mice for each group were used. Controls were administered vehicle and test groups were administered NSI-189 phosphate i.p. over 21 days as follows: D1-D7—3 mg/kg NSI-189 phosphate; D8-D14—10 mg/kg NSI-189 phosphate; D15-D21—30 mg/kg NSI-189 phosphate.

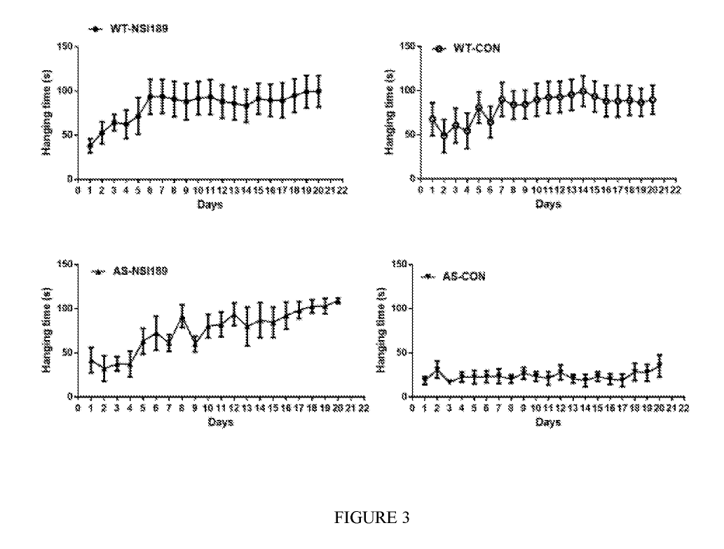

The results are shown in FIG. 3. As shown, the AS mice administered only vehicle were not able to hang onto the wire for more than about 20 seconds, whereas AS mice treated with NSI-189 ultimately matched the times of wild type mice at about 100 seconds.

Mice are also tested using the elevated body swing test (EBST) and Bederson exams. EBST is conducted by holding the mouse by its tail and recording the direction of swings in a clear Plexiglas® box (40×40×35.5 cm). Each rat is gently picked up at the base of the tail, and elevated until the nose is at a height of 2 inches (5 cm) above the surface. The direction of the swing, either left or right, is counted once the animals head moves sideways approximately 10 degrees from the midline position of the body. After a single swing, the animal is placed back in the Plexiglas® box and allowed to move freely for 30 seconds prior to retesting. These steps are repeated 20 times for each animal.

The Bederson exam is conducted using 3 tests: (1) forelimb retraction—measuring the ability of the rat to replace the forelimb after it is displaced laterally by 2 to 3 cm, graded from 0 (immediate replacement) to 3 (replacement after several seconds or no replacement); (2) beam walking ability, graded 0 for a rat that readily traverses a 2.4-cm-wide, 80-cm-long beam to 3 for a rat unable to stay on the beam for 10 seconds; and (3) bilateral forepaw grasp—the ability to hold onto a 2-mm-diameter steel rod, graded 0 for a rat with normal forepaw grasping behavior to 3 for a rat unable to grasp with the forepaws. The scores from all 3 tests, which can be done over a period of about 15 minutes on each assessment day, are added to give a mean neurologic deficit score (maximum possible score, 9 points divided by 3 tests=3).

1. A method to ameliorate cognitive deficiency and/or motor deficits associated with Angelman Syndrome (AS), which method comprises administering to a subject in need of such amelioration an effective amount of a 2-amino substituted nicotinamide or pharmaceutically acceptable salt thereof.

2. The method of claim 1 wherein the cognitive deficiency tested in laboratory models is a decrease in novel place recognition (NPR), a decrease in novel object recognition (NOR), a decrease in object in place (OiP) preference, a decrease in recognition of temporal order (TO) and/or an enhanced reaction to fear, and is tested in humans by the CogScreen test.

3. The method of claim 1 wherein the cognitive deficiency is a decrease in executive function and/or a decrease in attention and/or a decrease in memory and/or a decrease in working memory.

4. The method of claim 1 wherein the motor deficit is reflected in laboratory models in a Hanging Wire test, and in humans in a BMAT test.

5. The method of claim 1 wherein the 2-amino substituted nicotinamide is of the formula: 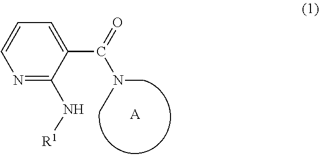
wherein R¹ is an alkyl of 3-8C and ring A is a 5- or 6-membered saturated ring optionally including an additional nitrogen which is unsubstituted or substituted with an additional nitrogen-containing substituent or a ring-opened form thereof.

6. The method of claim 5, wherein the 2-amino substituted nicotinamide is 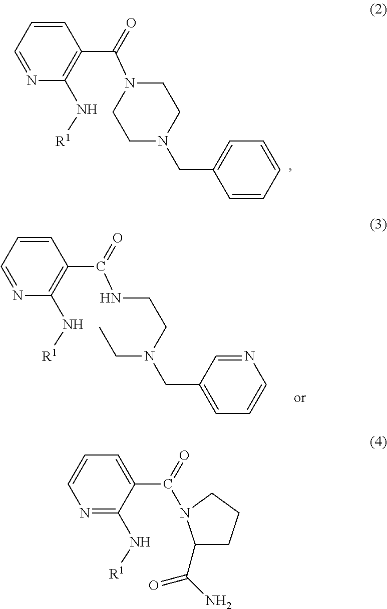
wherein R¹ is a branched alkyl group of 3-5C in formula (2) or (3) and is an alkyl group comprising a 5-6 membered saturated ring in formula (4).

7. The method of claim 6 wherein the 2-amino substituted nicotinamide is of formula (2) and R¹ is isoamyl.

8. The method of claim 1 wherein the 2-amino substituted nicotinamide is in the form a phosphate salt.

9. The method of claim 1 wherein said administering is oral or i.p.

10. The method of claim 1 which further includes subsequent testing of said subject for enhancement of said cognitive and/or motor skills.

11. The method of claim 10 wherein said testing for cognition is of a laboratory model and comprises novel place recognition (NPR) and/or novel object recognition (NOR) and/or object in place (OiP) and/or temporal order (TO).

12. The method of claim 10 wherein said testing for cognition is in humans and comprises a CogScreen test.

13. The method of claim 10 wherein said testing for motor skills is in humans and comprises a BMAT test.

14. The method of claim 10 wherein said testing for motor skills is in a laboratory model comprises a Hanging Wire test.

15. The method of claim 10 wherein said testing for cognitive comprises assessing executive function and/or attention and/or memory, and/or working memory.

16. The method of claim 8 wherein said administering is oral or i.p.

17. The method of claim 8 which further includes subsequent testing of said subject for enhancement of recovery of said cognition and/or motor skills.

18. The method of claim 15 wherein said testing is of a laboratory model and comprises novel place recognition (NPR) and/or novel object recognition (NOR) and/or object in place (OiP) and/or temporal order (TO).

19. The method of claim 13 wherein said testing comprises assessing executive function and/or attention and/or memory, and/or working memory.

20. The method of claim 18 wherein said testing for cognition is in humans and comprises a CogScreen test.

21. The method of claim 18 wherein said testing for motor skills is in humans and comprises a BMAT test.

22. The method of claim 18 wherein said testing is of a laboratory model and comprises the Hanging Wire test.
